(19) United States
(12) Patent Application Publication
(10) Pub. No.: US 2005/0234496 A1
(43) Pub. Date: Oct. 20, 2005

(54) Trocar assembly with cushioned activator

(76) Inventors: Timothy Wells, Ridgefield, CT (US); David Racenet, Litchfield, CT (US)

Correspondence Address: UNITED STATES SURGICAL,;A DIVISION OF TYCO HEALTHCARE GROUP LP, 150 GLOVER AVENUE, NORWALK, CT, 06856, US

(21) Appl. No.: 11/151,604

(22) Filed: Jun. 13, 2005

Related U.S. Application Data
(63) Continuation of application No. 09/865,774, filed on May 25, 2001.
(60) Provisional application No. 60/207,082, filed on May 25, 2000.

(51) Int. Cl. A61B 17/34

(52) U.S. Cl. 606/185

(57) ABSTRACT

A trocar assembly is provided which includes an obturator having a sharpened tip supported on one end thereof and a hand grip supported on an opposite end thereof. A cushioned grip member is supported on the hand grip. The cushioned grip member includes a slip resistant surface and is positioned on pressure contact regions of the hand grip.

This application claims priority from provisional application Ser. No. 60/207,082, filed May 25, 2000, which is incorporated herein by reference.

### BACKGROUND

1. Technical Field

The present disclosure relates generally to a surgical instrument for puncturing a body cavity. More particularly, the present disclosure relates to a trocar assembly for puncturing a body cavity having a hand grip including a cushioned slip-resistant portion.

2. Background of Related Art

Surgical instrumentation for puncturing body cavities, i.e., trocar assemblies are well known in the art. Typically, a trocar assembly includes an obturator having a sharpened tip at one end for piercing the body cavity and a hand grip portion mounted on the other end of the obturator which the surgeon grasps in the palm of his hand. The hand grip portion includes a plunger which engages the other end of the obturator and can be pressed with the palm of the hand to force the sharpened end of the obturator through the body cavity wall. Often, during endoscopic surgical procedures, multiple punctures through the body cavities are required.

In known trocar assemblies, the hand grip portion of the trocar assembly is formed from a hard plastic material and considerable force may be required to thrust the obturator through the body cavity wall. This force typically ranges from about 2 lbs. to about 20 lbs. and may be even higher, especially when operating on obese individuals. Such a force may cause discomfort to and eventually bruising of the surgeon's hand. Moreover, during most surgical procedures, blood and other body fluids collect on a surgeon's hands or gloves making it difficult for the surgeon to grip the hand grip portion of the trocar assembly.

Accordingly, a need exists for an improved trocar assembly which can be actuated by a surgeon without causing the surgeon discomfort and which can be securely gripped by a surgeon even in the presence of body fluids.

### SUMMARY

In accordance with the present disclosure, a trocar assembly is provided which includes an obturator having a sharpened tip at one end and a hand grip secured to the other end. The hand grip includes a cushioned slip resistant member. The cushioned member is preferably formed from a thermoplastic elastomer, e.g., Versaflex™ or Santaprene™, and over-molded onto the hand grip of the trocar assembly. Alternately, the cushioned member may be formed of other cushioned or pliant materials, e.g., elastomeric or synthetic materials, including isoprenes or nitrile or silicon containing material, etc. Moreover, the grip member can be fastened to the grip portion using other known fastening techniques, e.g., physical, chemical or mechanical, including adhesives, welding, screws, etc.

### BRIEF DESCRIPTION OF THE DRAWINGS

Various preferred embodiments of the presently disclosed trocar assembly are described herein with reference to the drawings, wherein:

FIG. 1 is a side cross-sectional view of the presently disclosed trocar assembly; and

FIG. 2 is a perspective view of one preferred embodiment of the presently disclosed trocar assembly positioned within a valved cannula assembly.

### DETAILED DESCRIPTION OF PREFERRED EMBODIMENTS

Preferred embodiments of the presently disclosed trocar assembly will now be described in detail with reference to the drawings in which like reference numerals designate identical or corresponding elements in each of the several views.

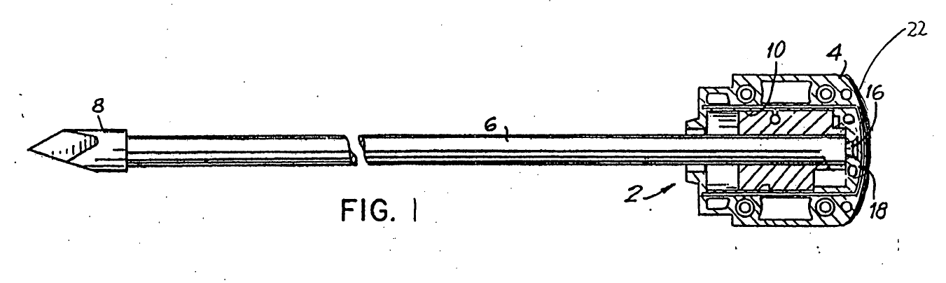

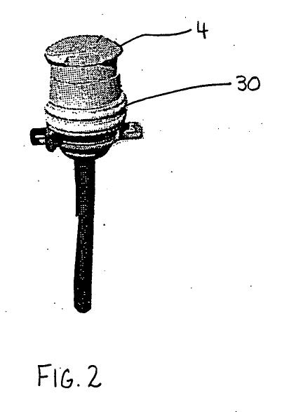

FIG. 1 illustrates a trocar assembly including an obturator 6 defining a longitudinal axis and having first and second ends. FIG. 2 illustrates the trocar assembly in combination with a cannula assembly 30. A sharpened tip 8 is mounted on the first end of the obturator 6. Tip 8 functions to penetrate or pierce a body cavity. A hand grip 4 is mounted on the second end of the obturator. Hand grip 4 is preferably formed from molded thermoplastic housing half-sections which are secured together to define a cavity 10 for receiving the second end of obturator 6. Alternately, other materials may be suitable for use, including other plastics, composites, surgical grade metals, etc.

A cushioned grip member 22 is secured to at least one pressure contact region of hand grip 4. The pressure contact regions of the hand grip include those areas of hand grip 4 to which a surgeon must grasp or apply pressure to during manipulation of the trocar assembly or insertion of obturator 6 through tissue into a body cavity. In a preferred embodiment, cushioned grip member 22 is formed from a thermoplastic elastomer or elastomer blend, such a Versaflex™ or Santoprene™, and is over-molded onto hand grip 4. A preferred thermoplastic elastomer is OM1040-X Versaflex™. Alternately, the use of different cushioned or pliant materials is envisioned, as is the use of different techniques for fastening grip member 22 onto hand grip 4. For example, grip member 22 may be formed from other pliant materials, including plastics, elastomers, synthetics, etc. Moreover, grip member 22 may be fastened to hand grip 4 using other fastening techniques, e.g., chemical, physical, or mechanical, including adhesives, screws, welding, interengaging members, bonding, fusing, coating, dipping, spraying, etc.

The use of a cushioned portion formed from a thermoplastic or an elastomeric material on the pressure contact regions of the handle assembly cushions the impact on a surgeon's hand during operation of the surgical instrument. Preferably, the cushioned portion is formed from a material having slip resistant properties which adhere well to the gloves worn by a surgeon, even in the presence of bodily fluids, to improve a surgeon's grip on the surgical instrument. In addition, the cushioned material may include a textured, roughened or ridged surface to enhance or provide the slip-resistant surface. The hardness of the cushioning material employed will vary depending on a particular surgical instrument and its application. The pressure required to actuate a particular instrument should be considered when choosing the material for forming the cushioned portion of the instrument. For example, a softer material may be more suitable for use with instruments requiring higher actuation pressures. Conversely, a harder material may be suitable for use in surgical instruments requiring smaller actuation pressures. The durometer of the cushioning material can be from about 10 to about 80, but is preferably between about 20 to about 50, and more preferably about 40.

Other factors should also be considered prior to selecting the cushioning material. These include whether the instrument is disposable or reusable and will be subjected to sterilization or other cleaning processes. If the instrument is reusable, a cushioning material having heat resistant properties should be used. In the alternative, it is contemplated that the cushioning member can be removable such that it could be removed from the surgical instrument prior to the sterilization and/or cleaning process. For example, the cushioning member could be provided as a removable flexible sleeve.

It will be understood that various modifications may be made to the embodiments disclosed herein. For example, it is envisioned that other pliant or cushion materials may be used to achieve a cushioning effect similar to that disclosed above. Moreover, the above described cushioned portion may be provided on other hand operated surgical devices. Therefore, the above description should not be construed as limiting, but hereby as exemplifications of preferred embodiments. Those skilled in the art will envision other modifications within the scope and spirit of the claims appended hereto.

1. A trocar assembly comprising: an obturator defining a longitudinal axis and having first and second ends, a sharpened tip positioned on the first end of the obturator and a hand grip positioned on a second end of the obturator opposite the first end, and a cushioned member of a pliant material positioned on at least one pressure contact surface of the hand grip, the cushioned member being arranged to cushion the impact on a surgeon's hand during use of the trocar assembly in forcing the sharpened tip of the obturator through a patient's body cavity wall.

2. A trocar assembly according to claim 1, wherein the cushioned member is formed from a thermoplastic elastomer.

3. A surgical instrument according to claim 2, wherein the cushioned member is over-molded onto the hand grip.

4. A trocar assembly according to claim 1, wherein the cushioned portion is formed from an elastomeric material.

5. A trocar assembly according to claim 1, wherein the cushioned member is secured to the hand grip with an adhesive.

6. A trocar assembly according to claim 1, wherein the cushioned member is secured to the hand grip by welding.

7. A trocar assembly according to claim 1, wherein the cushioned member is formed from a slip resistant material.

8. A trocar assembly according to claim 4, wherein the elastomeric material is selected from the group consisting of isoprene, nitrile and silicon.

9. A trocar assembly according to claim 1, wherein the hand grip is an elastomeric material.

10. A trocar assembly according to claim 1, wherein the cushioned member is over-molded onto the hand grip.
